(19) United States
(12) Patent Application Publication
(10) Pub. No.: US 2009/0325255 A1
(43) Pub. Date: Dec. 31, 2009

(54) PROCESS FOR OVER-PRODUCTION OF HYDROGEN

(75) Inventors: Tapan Chakravarti, Maharashtra (IN); Suresh Kumar Manukonda, Maharashtra (IN); Atul Narayanrao Vaidya, Maharashtra (IN); Sandeep Narayan Mudliar, Maharashtra (IN); Sukumar Devotta, Maharashtra (IN); Banibrata Pandey, Panjagutta Hyderabad (IN); Pidaparti Seshasadri Sastry, Hyderabad (IN)

Correspondence Address: BANNER & WITCOFF, LTD., TEN SOUTH WACKER DRIVE, SUITE 3000, CHICAGO, IL, 60606, US

(73) Assignee: NAGARJUNA ENERGY PRIVATE LIMITED, Hyderabad (IN)

(21) Appl. No.: 12/279,232

(22) PCT Filed: Feb. 13, 2007
(86) PCT No.: PCT/IB07/00327

§ 371 Date: Apr. 20, 2009

(30) Foreign Application Priority Data
Feb. 13, 2006 (IN) 1127/MUM/2006

(51) Int. Cl. C12P 3/00; C12M 1/107; C25B 1/02

(52) U.S. Cl. 435/168; 435/300.1; 205/637

(57) ABSTRACT

The present invention provides a process of increasing production of hydrogen during fermentation process and also an electro-biochemical is designed to achieve higher hydrogen production.

### FIELD OF THE PRESENT INVENTION

The present invention is in the field of hydrogen production.

### BACKGROUND AND PRIOR ART

The excessive burning of fossil fuels which results in the generation of CO₂, Soₓ, and Noₓ is one of the primary causes of global warming and acid rain, which have started to affect the earth's climate, weather, vegetation and aquatic ecosystems. Hydrogen is the cleanest energy source, producing water as its only combustion product. Hydrogen can be produced from renewable raw materials such as biomass and water. Therefore, hydrogen is a potential clean energy substitute for fossil fuels. Despite the “green” nature of hydrogen as a fuel, it is still primarily produced from nonrenewable sources such as natural gas and petroleum based hydrocarbons via steam reforming, and only 4% is generated from water using electrolysis. However these processes are highly energy-intensive and not always environmentally benign. Given these perspectives, biological hydrogen production assumes paramount importance as an alternative energy source.

Fermentation of biomass or carbohydrate-based substrates presents a promising route of biological hydrogen production, compared with photosynthetic or chemical routes. Pure substrates, including glucose, starch and cellulose, as well as different organic waste materials can be used for hydrogen fermentation. Among a large number of microbial species, strict anaerobes and facultative anaerobic chemoheterotrophs, such as clostridia and enteric bacteria, are efficient producers of hydrogen. Despite having a higher evolution rate of hydrogen, the yield of hydrogen is 4 moles H₂ per mole of glucose using fermentative processes is lower than that achieved using other methods; thus, the process is not economically viable in its present form. The pathways and experimental evidence cited in the literature reveal that a maximum of four mol of hydrogen can be obtained from substrates such as glucose.

Fermentation of glucose by all known microbiological routes can produce theoretically up to 4 mol of hydrogen per mol of glucose. 96.7% conversion efficiency based on 4 moles of H₂/mol Glucose was achieved by researcher only by using enzymes.

The main challenge to fermentative production of hydrogen is that only 15% of the energy from the organic source can typically be obtained in the form of hydrogen. While a conversion efficiency of 33% is theoretically possible for hydrogen production from glucose (based on maximum four moles hydrogen per mole glucose), only half of this is usually obtained under batch and continuous fermentation conditions. Four moles of hydrogen could only be obtained from glucose if two moles of acetate are produced, however only two moles of hydrogen are produced when butyrate is the main fermentation product. Typically, 60-70% of the aqueous product during sugar fermentation is butyrate. This is because high H₂ pressure inside the reactor results in the inhibition of pyruvate ferrodoxin oxidoreductase and pyruvate formate lyase, the two enzymes responsible for conversion of pyruvate to acetate. Thus a low hydrogen pressure of around 10⁻³ atm is necessary for achieving high conversion efficiency.

A thermophilic organism has recently been reported that may be able to achieve higher conversion efficiencies. However, its biochemical route of hydrogen production is unknown, and claims of high hydrogen production conversion have not been independently verified or shown to be economical.

Genetic engineering of bacteria could increase hydrogen recovery. However, even if biochemical pathways that are used by bacteria such as Clostridia are successfully modified to increase hydrogen production by optimizing the production of acetate, the maximum conversion efficiency will still remain below 33%.

In view of the above said draw back, Applicant has made an effort to develop a method results in higher production of hydrogen from glucose.

### OBJECTIVE OF THE PRESENT INVENTION

The object of the present invention is to develop a method to increase production of hydrogen in a fermentation process.

Yet in another object of the present invention is to develop a reactor to implement the above method.

Abbreviation used in the Application

VFA=Volatile fatty acids

### BRIEF DESCRIPTION OF FIGURES

FIG. 1 Schematic representation of the electro biochemical reactor with electrodes for capturing protons released during anaerobic fermentation.

### DETAILED DESCRIPTION OF THE PRESENT INVENTION

Accordingly, the present invention reveals a process of increasing production of hydrogen of a fermentation process. In order to achieve the same, an electro-biochemical reactor is developed to capture protons by applying electrical charge, which is generated during acidogenic phase of fermentation.

As evident from prior art on fermentative hydrogen production, the yield of hydrogen is low and the reason behind this is higher partial pressure of hydrogen. Higher yield requires maintaining of low partial pressure of hydrogen in the reactor to make the reaction thermodynamically favorable towards conversion of pyruvate to acetate and not to other reduced end products such as butyrate. Also the protons formed during fermentation lower the pH of the fermentation broth, thereby reducing the rate of hydrogen production. Various strategies (e.g. nitrogen sparging) have been reported for hydrogen removal. Most of these approaches further require separation of hydrogen from the stripping inert gas thereby increasing the hydrogen production cost. However, none of the prior art has given any clue to capture the excess proton and convert those to molecular hydrogen and there by increase the conversion ratio of hydrogen from substrate.

The protons generated in the fermentative broth is converted to hydrogen at negatively charged electrode and if simultaneously removed, will not only enable the system in maintaining low partial pressure of hydrogen and constant pH but also increase the quantity of hydrogen production.

This in turn enhances the rate of hydrogen production as a result of low hydrogen partial pressure by activating two hydrogen repressed enzymes such as pyruvate-ferredoxin oxidoreductase and pyruvate-formate lyase which convert pyruvate to acetate, an essential pre-requisite for generating four moles of hydrogen per mole of glucose.

The present invention suggests a system, whereby the proton generated during acidogenic phase in an anaerobic process can be converted to hydrogen and thereby increases the yield of hydrogen in heterotrophic fermentation. Therefore the yield of hydrogen will be higher than the stoichiometrically possible maximum yield.

Following is the reaction takes place during breakdown of glucose in Heterotrophic fermentation (HF)

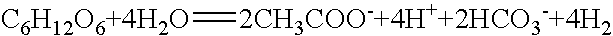

The above reaction in an anaerobic fermentor clearly indicates that 4 moles of molecular hydrogen can be obtained from 1 moles of glucose. The method of the present invention traps the excess proton (4H⁺) and converts them into molecular hydrogen there by increasing the yield.

The said four protons (4H⁺) are captured during a transition phase just before formation of acetic acid. The two protons are the counterpart of acetate ions and remaining two are of bicarbonate ions. Under normal circumstances and conventional fermentation process, the free protons combine with acetate ion to form acetic acid and with bi-carbonate finally to form H₂O and CO₂. Upon applying electric current the free protons are converted to molecular hydrogen, which is then taken into gas collection chamber. By capturing protons, low atmospheric pressure of hydrogen is maintained during the anaerobic fermentation, which in turn helps the microorganism to activate pyruvate ferrodoxin oxidoreductase and pyruvate formate-lyase.

The following schematic diagram represents a schematic diagram that explains the source of protons and mechanism of converting those protons into molecular hydrogen. An unstable phase i.e. Just before the formation of acetic acid, CH₃COO⁻ and 2HCO₃⁻ get generated. Since the ionic state is very unstable, these negatively charged ions tend to combine with protons to acetic acid. Present invention proposes to capture these protons to prevent formation of acetic acid and subsequently those protons are converted to molecular hydrogen upon application of mild electric current. There has been no decrease in the acetic acid concentration, which indicates that H⁺ ions are not generated due to break down of acetic acid but just before the formation of acetic acid during fermentation process.

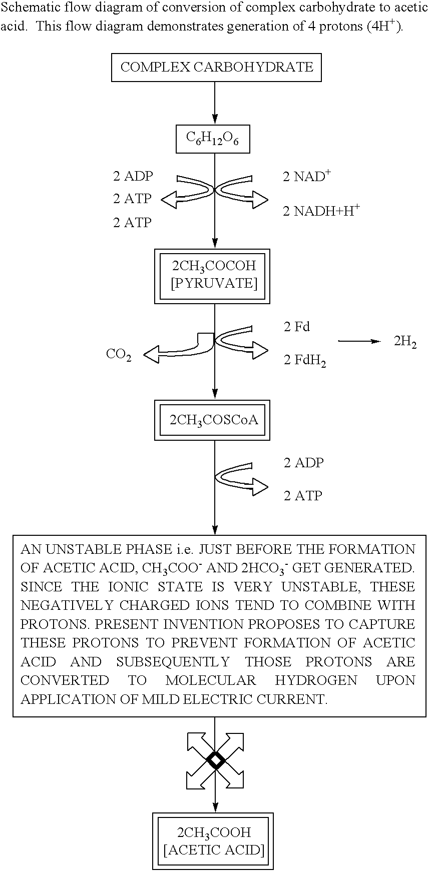

Accordingly the present invention provides a process for over-production of hydrogen in a heterotrophic fermentation process, said process comprising the steps:
a) culturing microorganism in a nutrient medium under anaerobic condition and allow to proceed fermentation at a temperature in the range of 25 to 40° C. for a period of 36 to 72 hours in a fermentor including charged electrodes, and
b) capturing protons generated during fermentation by applying an electric charge to the electrode and selectively attracting the protons to the electrode to produce molecular hydrogen and collecting the same along with the hydrogen produced by the microorganism during fermentation.

In another embodiment of the present invention, the temperature is 37° C.

Still in another embodiment of the present invention, the nutrient medium is selected from a group comprising sugar and fermentable organic acids.

Yet in another embodiment of the present invention the sugar is selected from a group comprising hexose, pentose.

The invention further provides to a bio-reactor used for heterotrophic fermentation process, said bioreactor comprising:
a) a vessel for fermentation,
b) at least one electrode, the electrode adapted to selectively capture desired charged particle when potentialized,
c) an outlet to collect the gas, and
d) optionally comprising a means to store produced hydrogen.

In one more embodiment of the present invention is related to a method of trapping excess charged particles from a fermentor produced during bio-chemical reaction in a fermentor, said method comprising introducing into the fermentor an electrode, capturing charged particle by applying an electric charge to the electrode and selectively attracting the desired charged particles to the electrode and trapping the same from the encapsulated electrode.

Further, in another embodiment of the present invention, the electrode can optionally be encapsulated by gas permeable membrane

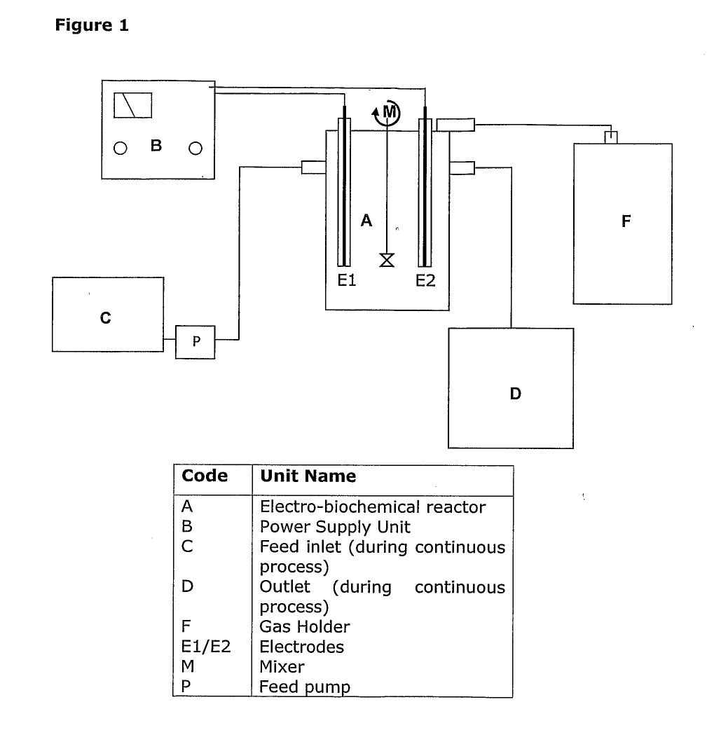

FIG. 1 shows an electro-biochemical reactor [A] for enhanced hydrogen production by capturing the protons released during anaerobic fermentation/digestion and simultaneous removal of hydrogen from the system, which comprises of a fermentor containing two electrodes [E1] and [E2] connected to electric potential [B] (in DC) for proton capture at the negatively charged electrode or cathode, and a gas collector [F] for collection of hydrogen generated at negatively charged electrode. [C] represents the feed pump inlet, while [D] represents the outlet for collecting spent medium. The C and D are used only in continuous fermentation. A pump can also be used to collect gas produced in the reactor.

TABLE 1
Production of Hydrogen by Clostridium sp. ATCC824 along
with % age increase of hydrogen as compared to control.

| | | Glucose | | % increase |
|---|---|---|---|---|
| Set of | | Consumption | Yield of H₂ (mol)/ | H₂ (mol)/ |
| Exps. | | (gm/L) | Glucose (mol) | Glucose (mol) |
| I | C | 3.48 | 1.30 | |
| | E | 4.32 | 1.72 | 32.30 |
| II | C | 3.51 | 1.32 | |
| | E | 4.48 | 1.67 | 26.51 |
| III | C | 2.66 | 1.25 | |
| | E | 3.4 | 1.68 | 34.40 |

C = Control (medium + culture)
E = Experiment (medium, culture and electrode)

TABLE 2
Production of Hydrogen by Clostridium cellulovoron
BSMZ3052 along with % age increase of hydrogen as
compared to control.

| | | Suger | | % increase |
|---|---|---|---|---|
| Set of | | Consumption | Yield of H₂ (mol)/ | H₂ (mol)/ |
| exps. | | (gm/L) | Glucose (mol) | Glucose (mol) |
| I | C | 4.23 | 1.58 | |
| | E | 5.92 | 2.13 | 34.81 |
| II | C | 6.78 | 1.62 | |
| | E | 9.35 | 2.21 | 36.41 |
| III | C | 5.80 | 1.70 | |
| | E | 8.23 | 2.33 | 37.05 |

C = Control (containing medium + culture)
E = Experiment (medium, culture and electrode)

### Examples

The following examples are given by way of illustration of the working of the invention in actual practice and therefore should not be construed to limit the scope of the present invention.

### Example 1

Medium Composition:

Media used for growth and biomass generation of the cultures used in the present invention is having the following ingredients:

| | | |
|---|---|---|
| Beaf extract | 45 | g/l |
| Peptone | 20 | g/l |
| Dextrose | 2 | g/l |
| NaCl | 5 | g/l |
| Crystalline HCl | 0.5 | g/l |
| Distilled water | 1000 | ml |

Media composition used for hydrogen production comprising following ingredients:

| | | |
|---|---|---|
| Protease peptone | 5 | g/l |
| KH2PO4 | 2 | g/l |
| Yeast extract | 0.5 | g/l |
| MgSO4•7H2O | 0.5 | g/l |
| L-cystine HCL | 1 | g/l |
| Dextrose | 10 | g/l |
| Distilled water | 1000 | ml |

### Example 2

One liter of sterilized media containing 20 g/l glucose with necessary nutrients and inoculated with pure culture of clostridium species, were subjected to anaerobic fermentation in a 2 liter fermentor at constant temperature of 30° C. One litre of sterilized media containing 20 g/l glucose with necessary nutrients and inoculated with pure culture of clostridium specie bearing accession number Clostridium sp. ATCC824 and Clostridium cellulovoron BSMZ3052 were subjected to anaerobic fermentation in a 2 liter electro biochemical reactor (FIG. 1) at constant temperature of 30° C. The applied cathode potential was between 2.0 and 4 V, while the current density was 0.3 and 3.0 mA. The total fermentation time was 48 hrs and the total gas produced was collected in a conventional gas collection system based liquid displacement technique. Gas was analyzed for hydrogen content using Gas chromatograph (electron capture detector) on parapak Q SS column.

A parallel control experiment was carried out without electrode i.e. using conventional fermentor and the same microorganism used in the experiments to assess the efficacy of proton capture as disclosed in the instant application. Also, fermentation was carried out only with electrodes using medium used in the experiment but without culture to find out whether H₂ is getting generated because of applying current to medium (refer Table 1). Since, hydrogen production was negligible; the Applicant did not carry out further experiments with medium and electrodes.

From the above examples it can be noted that the electro-biochemical system can be used for enhanced production of hydrogen by capturing proton released during anaerobic fermentation/digestion of various substrates under low hydrogen pressure of around 10⁻³ atm. Proton capture at cathode will play a duel role; the capture will enhance hydrogen production and maintain the pH at near neutral (around 7.0) condition. An intersecting feature of the present invention is the use of charged electrodes for the capture of protons generated during anaerobic fermentation/digestion of various substrates for the enhanced production of hydrogen using mutated cultures where enzymes converting pyruvate to acetate are insensitive to hydrogen as compared to conventional fermentative hydrogen production, which is limited due to lowering of pH and accumulation of hydrogen. Also the purity of hydrogen gas obtained from electro biochemical reactor is high as compared to that produced from conventional anaerobic fermentation.

Advantages
1. Enhanced hydrogen production compared to conventional anaerobic fermentative processes due to capture the protons generated during anaerobic digestion of various substrates & maintenance of pH at around 7.0 that prevents excessive acidity in fermentation broth.
2. Capture of protons generated from the fermentation broth will thus help in maintaining the pH without addition of alkali and also results in increase in the rate of the reaction.
3. The electro-biochemical reactor maintained at a low hydrogen pressure of around 10⁻³ atm can be used for enhanced hydrogen production via proton capture during anaerobic fermentation as well as anaerobic digestion of various substrates.
4. Use of mixed consortium of microorganisms makes the process easy to operate and there is no need of sterilization of the substrate as compared to pure fermentative microorganisms

1. A process for overproduction of hydrogen in a heterotrophic fermentation process, said process comprising the steps:
a. culturing microorganism in a nutrient medium under anaerobic condition and allow to proceed fermentation at a temperature in the range of 25 to 40° C. for d period of 36 to 72 hours in a fermentor including charged electrodes, and
b. capturing protons generated during fermentation by applying an electric charge to the electrode and selectively attracting the protons to the electrode to produce molecular hydrogen and collecting the same along with the hydrogen produced by the microorganism during fermentation.

2. A process as claimed in claim 1, wherein in step (a) the temperature is 37° C.,

3. A process as claimed in claim 1, wherein the nutrient medium is selected from a group comprising sugar and fermentable organic acids.

4. A process as claimed in claim 3, wherein the sugar is selected from a group comprising hexose, pentose.

5. A bioreactor used for heterotrophic fermentation process, said bioreactor comprising:
a. a vessel for fermentation,
b. at least one electrode, said electrode adapted to selectively capture desired charged particle when potentialized,
c. an outlet to collect the gas, and
d. optionally comprising a means to store produced hydrogen.

6. A method of capturing protons from a fermentor produced during fermentation process of claim 1, said method comprising introducing into the fermentor at least one electrode, capturing charged particle by applying an electric charge to the electrode and selectively attracting the desired charged particles to the electrode and capturing the said particle.
